(19) United States
(12) Patent Application Publication
(10) Pub. No.: US 2013/0150408 A1
(43) Pub. Date: Jun. 13, 2013

(54) SUBSTITUTED DIOXOPIPERIDINYL PHTHALIMIDE DERIVATIVES

(71) Applicant: Concert Pharmaceuticals, Inc., Lexington, MA (US)

(72) Inventors: Julie F. Liu, Lexington, MA (US); Roger D. Tung, Lexington, MA (US)

(73) Assignee: CONCERT PHARMACEUTICALS, INC., Lexington, MA (US)

(21) Appl. No.: 13/762,060

(22) Filed: Feb. 7, 2013

Related U.S. Application Data
(63) Continuation of application No. PCT/US2011/064238, filed on Dec. 9, 2011.
(60) Provisional application No. 61/421,777, filed on Dec. 10, 2010.

(51) Int. Cl. A61K 31/454; C07D 401/04

(52) U.S. Cl. 514/323; 546/198

(57) ABSTRACT

This invention relates to novel substituted dioxopiperidinyl phthalimide derivatives and pharmaceutically acceptable acid addition salts thereof. The invention also provides compositions comprising a compound of this invention and the use of such compositions in methods of treating diseases and conditions beneficially treated by an immunomodulatory agent.

### RELATED APPLICATIONS

This application is claims the benefit of U.S. Provisional Patent Application No. 61/421,777, filed Dec. 10, 2010, the entire contents of which are hereby incorporated by reference.

### BACKGROUND

Many current medicines suffer from poor absorption, distribution, metabolism and/or excretion (ADME) properties that prevent their wider use. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated decreased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

This invention relates to novel substituted dioxopiperidinyl phthalimide derivatives and pharmaceutically acceptable salts thereof. The invention also provides compositions comprising a compound of this invention and the use of such compositions in methods of treating diseases and conditions beneficially treated by an immunomodulatory agent.

Pomalidomide, chemically known as 4-amino-2-(2,6-dioxopiperidin-3-yl)-1,2-dihydroisoindole-1,3-dione and its pharmaceutically acceptable salts thereof are disclosed as immunomodulatory agents. Pomalidomide has been shown to suppress the production of tumor necrosis factor alpha (TNF-α). Pomalidomide has demonstrated usefulness in the treatment of primary myelofibrosis (J Clin Oncol 2009, 27(27): 4563, Thomson Reuters Drug News (formerly DailyDrugNews.com) Aug. 10, 2010) as well as in the treatment of relapsed or refractory multiple myeloma when used alone or in combination with dexamethasone (J Clin Oncol 2009, 27(30): 5008, 50th Annu Meet Am Soc Hematol (December 6-9, San Francisco) 2008).

Pomalidomide is also in clinical trials, alone or in combination with other therapeutic agents, for the treatment of multiple myeloma, Waldenstrom's macroglobulinemia, small cell lung cancer, graft-versus host disease, chronic cough in patients with idiopathic pulmonary fibrosis, pancreatic cancer and soft tissue sarcoma.

Pomalidomide is associated with significant potential toxicities, which include human birth defects; neutropenia; moderate to severe bone marrow suppression in patients with myelofibrosis; and dyspnea.

Despite the beneficial activities of pomalidomide, there is a continuing need for new compounds to treat the aforementioned diseases and conditions.

### DEFINITIONS

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” is meant any condition or disorder that damage or interferes with the normal function of a cell, tissue, or organ.

It will be recognized that some variation of natural isotopic abundance occurs in a synthesized compound depending upon the origin of chemical materials used in the synthesis. Thus, a preparation of pomalidomide will inherently contain small amounts of deuterated isotopologues. The concentration of naturally abundant stable hydrogen isotopes, notwithstanding this variation, is small and immaterial with respect to the degree of stable isotopic substitution of compounds of this invention. See for instance Wada, E and Hanba, Y, Seikagaku, 1994, 66: 15; Gannes, L Z et al, Comp Biochem Physiol A Mol Integr Physiol, 1998, 119: 725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

The compounds of the present invention contain one or more asymmetric carbon atoms. As such, a compound of this invention can exist as the individual enantiomers as well a mixture of enantiomers. Accordingly, a compound of the present invention will include not only a racemic mixture, but also individual respective enantiomers substantially free of other enantiomers. The term “substantially free of other enantiomers” as used herein means less than 25% of other enantiomers, preferably less than 10% of other enantiomers, more preferably less than 5% of other enantiomers and most preferably less than 2% of other enantiomers are present. Methods of obtaining or synthesizing enantiomers are well known in the art and may be applied as practicable to final compounds or to starting material or intermediates.

Unless otherwise indicated when a disclosed compound is named or depicted by a structure without specifying the stereochemistry and has one or more chiral centers, it is understood to represent all possible stereoisomers of the compound.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert and “t-” each refer to tertiary. “US” refers to the United States of America.

Throughout this specification, the terms “each Z,” and “each W” mean, all “Z” groups (e.g., Z¹, Z², Z³, Z⁴ and Z⁵), and all “W” groups (e.g., W¹, W² and W³), respectively.

### Therapeutic Compounds

According to one embodiment, the present invention provides a compound of Formula I:

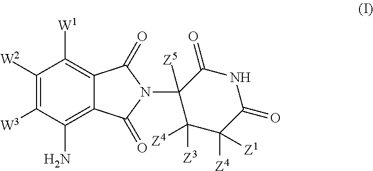

or a pharmaceutically acceptable salt thereof, wherein:

each W is independently selected from hydrogen and deuterium;

each Z is independently selected from hydrogen and deuterium; and

at least one W or one Z is deuterium.

In one embodiment, Z⁵ is deuterium. In one aspect of this embodiment, Z³ and Z⁴ are each hydrogen. In another aspect of this embodiment, Z³ and Z⁴ are each deuterium. In one aspect of this embodiment, Z¹ and Z² are each hydrogen. In one aspect of this embodiment, Z¹ and Z² are each deuterium.

In one aspect of the embodiment wherein Z⁵ is deuterium, Z³ and Z⁴ are each deuterium; Z¹ and Z² are each hydrogen; and W¹, W² and W³ are each hydrogen or each deuterium.

In one aspect of the embodiment wherein Z⁵ is deuterium, Z³ and Z⁴ are each deuterium; Z¹ and Z² are each deuterium; and W¹, W² and W³ are each hydrogen or each deuterium.

In one aspect of the embodiment wherein Z⁵ is deuterium, Z¹, Z², Z³ and Z⁴ are each hydrogen; and W¹, W² and W³ are each hydrogen or each deuterium.

In one embodiment, W¹, W² and W³ are the same. In one aspect of this embodiment W¹, W² and W³ are simultaneously deuterium. In another aspect of this embodiment W¹, W² and W³ are simultaneously hydrogen.

In another embodiment, each Z attached to a common carbon atom (that is, either Z¹ and Z² or Z³ and Z⁴) is the same. In one aspect of this embodiment, each member of at least one pair of Z attached to a common carbon atom is deuterium. In another aspect of this embodiment, Z¹, Z², Z³ and Z⁴ are simultaneously deuterium. In another aspect of this embodiment, Z¹, Z², Z³, Z⁴ and Z⁵ are simultaneously deuterium. In still another aspect, Z¹, Z², Z³, Z⁴ and Z⁵ are simultaneously deuterium and W¹, W² and W³ are simultaneously hydrogen.

In another set of embodiments, any atom not designated as deuterium in any of the embodiments set forth above is present at its natural isotopic abundance.

In another embodiment, the compound is selected from any one of the compounds set forth below:

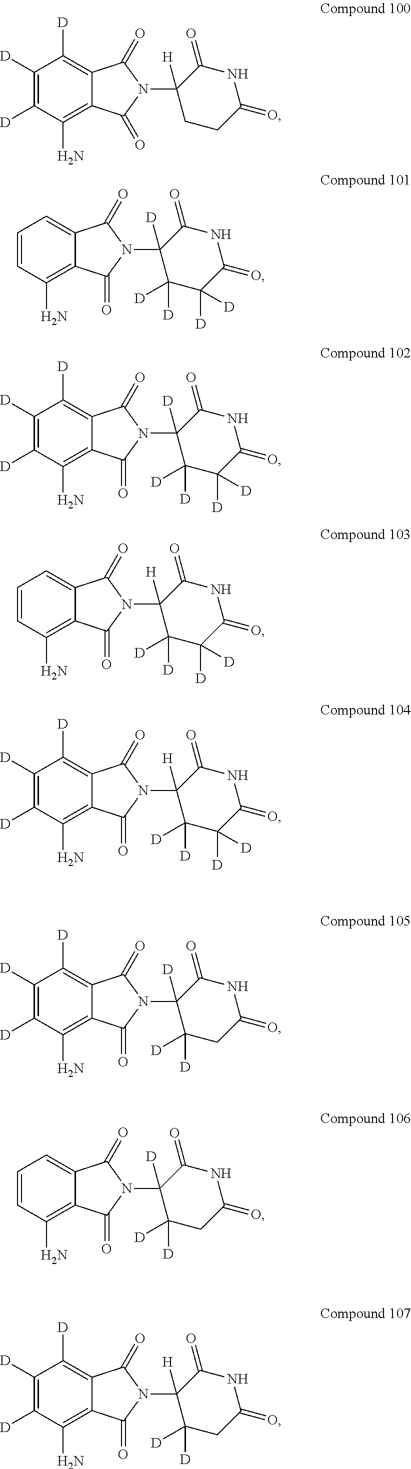

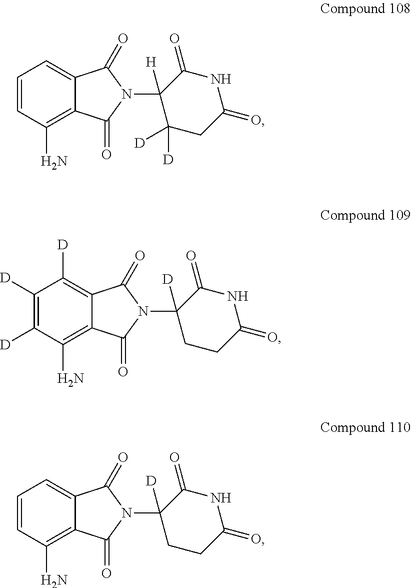 [SMILES: [2H]C1([2H])CC(=O)NC(=O)C1N1C(=O)c2cccc(N)c2C1=O ; [2H]c1c([2H])c(N)c2c(c1[2H])C(=O)N(C1([2H])CCC(=O)NC1=O)C2=O ; [2H]C1(N2C(=O)c3cccc(N)c3C2=O)CCC(=O)NC1=O]

and a pharmaceutically acceptable salt thereof, wherein any atom not designated as deuterium is present at its natural isotopic abundance.

In one embodiment, the invention provides a compound of Formula I which is a compound of Formula Ia or Ib:

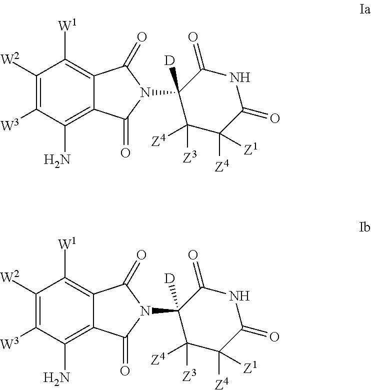

wherein W and Z are as defined above.

In one aspect of this embodiment, Z³ and Z⁴ are each hydrogen. In another aspect of this embodiment, Z³ and Z⁴ are each deuterium. In one aspect of this embodiment, Z¹ and Z² are each hydrogen. In one aspect of this embodiment, Z¹ and Z² are each deuterium.

In one aspect of this embodiment, Z³ and Z⁴ are each deuterium; Z¹ and Z² are each hydrogen; and W¹, W² and W³ are each hydrogen or each deuterium.

In one aspect of this embodiment, Z³ and Z⁴ are each deuterium; Z¹ and Z² are each deuterium; and W¹, W² and W³ are each hydrogen or each deuterium.

In one aspect of this embodiment, Z¹, Z², Z³ and Z⁴ are each hydrogen; and W¹, W² and W³ are each hydrogen or each deuterium.

In one aspect of this embodiment, W¹, W² and W³ are the same. In one aspect of this embodiment W¹, W² and W³ are simultaneously deuterium. In another aspect of this embodiment W¹, W² and W³ are simultaneously hydrogen.

In another aspect of this embodiment, each Z attached to a common carbon atom (that is, either Z¹ and Z² or Z³ and Z⁴) is the same. In one aspect of this embodiment, each member of at least one pair of Z attached to a common carbon atom is deuterium. In one more particular aspect of this embodiment, Z¹, Z², Z³, and Z⁴ are simultaneously deuterium and W¹, W² and W³ are simultaneously hydrogen.

Compounds of Formula Ia and Ib may be obtained from compounds of formula I, for example, by chiral HPLC separation.

The rate of epimerization for a compound of Formula Ia or Ib, as compared to the corresponding enantiomer of pomalidomide, can be readily measured using techniques well known to the skilled artisan. For example, pure samples of compounds of Formula Ia and Ib as well as pure samples of each enantiomer of pomalidomide can be isolated and analyzed using chiral HPLC. These pure samples can be dissolved to an appropriate concentration in an appropriate physiological buffer or bodily fluid or simulant thereof and monitored over time (for example, approximately every 5 minutes) using chiral HPLC, to assess the rate of epimerization.

In a further embodiment, the compound is selected from any one of the compounds set forth below:

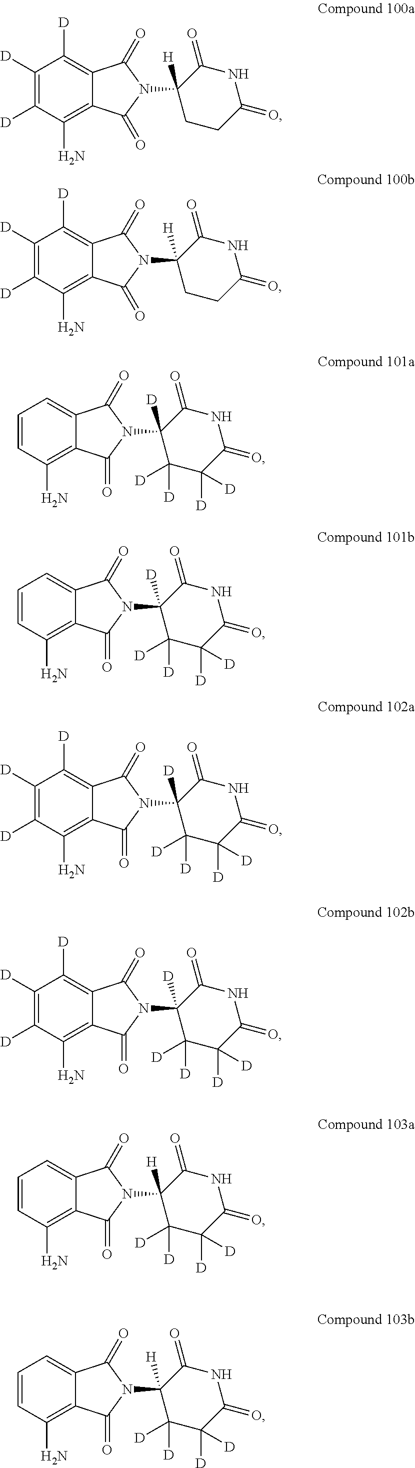

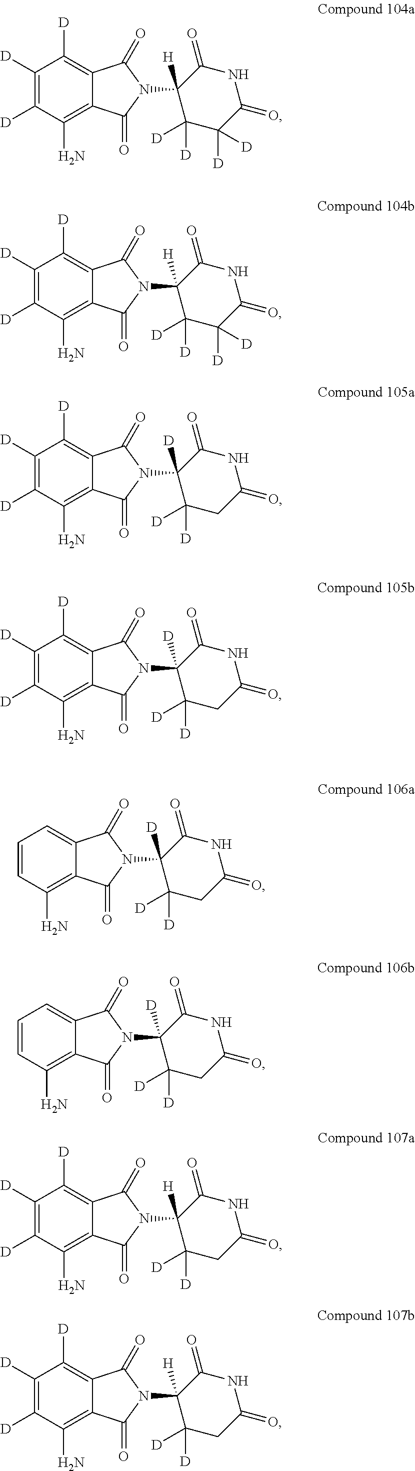

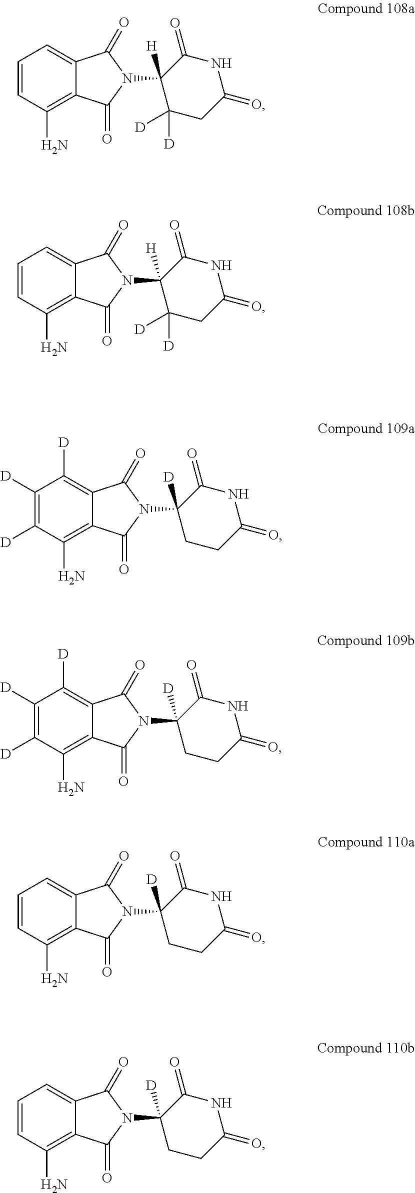 [SMILES: [2H]C1([2H])CC(=O)NC(=O)[C@H]1N1C(=O)c2cccc(N)c2C1=O ; [2H]C1([2H])CC(=O)NC(=O)[C@@H]1N1C(=O)c2cccc(N)c2C1=O ; [2H]c1c([2H])c(N)c2c(c1[2H])C(=O)N([C@@]1([2H])CCC(=O)NC1=O)C2=O ; [2H]c1c([2H])c(N)c2c(c1[2H])C(=O)N([C@]1([2H])CCC(=O)NC1=O)C2=O ; [2H][C@]1(N2C(=O)c3cccc(N)c3C2=O)CCC(=O)NC1=O ; [2H][C@@]1(N2C(=O)c3cccc(N)c3C2=O)CCC(=O)NC1=O]

and a pharmaceutically acceptable salt thereof, wherein any atom not designated as deuterium is present at its natural isotopic abundance.

In one embodiment of Compound 101a or 101b, or a pharmaceutically acceptable salt thereof, the isotopic enrichment factor for the deuterium atom bonded to the carbon indicated with “Cₐ” in the figure below (shown for 101a) is at least 5500 (82.5% deuterium incorporation), at least 6000 (90% deuterium incorporation), or at least 6333.3 (95% deuterium incorporation);

the isotopic enrichment factor for the deuterium atoms bonded to the carbon indicated with “C_{b}” in the figure below is at least 5500 (82.5% deuterium incorporation), at least 6000 (90% deuterium incorporation), or at least 6333.3 (95% deuterium incorporation);

and the isotopic enrichment factor for the deuterium atom bonded to the carbon indicated with “C_{c}” in the figure below is at least 5500 (82.5% deuterium incorporation), at least 6000 (90% deuterium incorporation), or at least 6333.3 (95% deuterium incorporation):

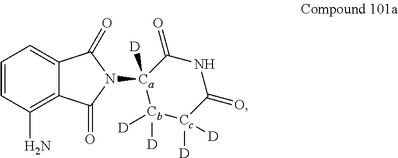

wherein any atom not designated as deuterium is present at its natural isotopic abundance.

In one embodiment of Compound 106a or 106b, or a pharmaceutically acceptable salt thereof, the isotopic enrichment factor for the deuterium atom bonded to the carbon indicated with “Cₐ” in the figure below (shown for 106a) is at least 5500 (82.5% deuterium incorporation), at least 6000 (90% deuterium incorporation), or at least 6333.3 (95% deuterium incorporation); and the isotopic enrichment factor for the deuterium atoms bonded to the carbon indicated with “C_{b}” in the figure below is at least 5500 (82.5% deuterium incorporation), at least 6000 (90% deuterium incorporation), or at least 6333.3 (95% deuterium incorporation):

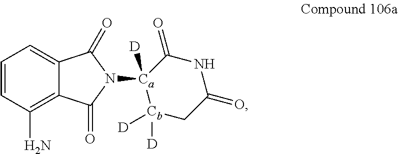

wherein any atom not designated as deuterium is present at its natural isotopic abundance.

In one embodiment of Compound 105a or 105b, or a pharmaceutically acceptable salt thereof, the isotopic enrichment factor for the deuterium atom bonded to the carbon indicated with “Cₐ” in the figure below (shown for 105a) is at least 5500 (82.5% deuterium incorporation), at least 6000 (90% deuterium incorporation), or at least 6333.3 (95% deuterium incorporation);

the isotopic enrichment factor for the deuterium atoms bonded to the carbon indicated with “C_{b}” in the figure below is at least 5500 (82.5% deuterium incorporation), at least 6000 (90% deuterium incorporation), or at least 6333.3 (95% deuterium incorporation);

and the isotopic enrichment factor for the deuterium atom bonded to each carbon indicated with “Cₑ” in the figure below is at least 5500 (82.5% deuterium incorporation), at least 6000 (90% deuterium incorporation), or at least 6333.3 (95% deuterium incorporation);

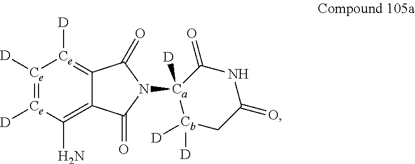

wherein any atom not designated as deuterium is present at its natural isotopic abundance.

In one embodiment of Compound 110a or 110b or a pharmaceutically acceptable salt thereof, the isotopic enrichment factor for the deuterium atom bonded to the carbon indicated with “Cₐ” in the figure below (shown for 110a) is at least 5500 (82.5% deuterium incorporation), at least 6000 (90% deuterium incorporation), or at least 6333.3 (95% deuterium incorporation):

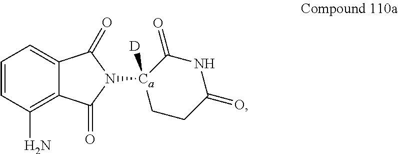

wherein any atom not designated as deuterium is present at its natural isotopic abundance.

In one embodiment the invention is directed at a compound of the formula II

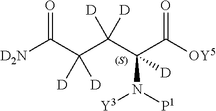

wherein Y³ is hydrogen or deuterium, Y⁵ is hydrogen or deuterium, and P¹ is a protecting group. In one embodiment, Y³ is hydrogen. In one embodiment, Y³ is deuterium. In one embodiment, Y⁵ is hydrogen. In one embodiment, Y⁵ is deuterium. In one embodiment, P¹ is a group of the formula —C(O)-Q-R², wherein Q is O or NH, and R² is (a) C₁-C₆ alkyl optionally substituted with C₆-C₁₀ aryl; (b) C₃-C₈ cycloalkyl; or (c) C₆-C₁₀ aryl. In one more particular embodiment, P¹ is benzyloxycarbonyl.

In one embodiment the invention is directed at a compound of the Formula III

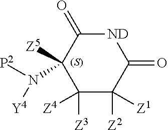

wherein Z¹-Z⁵ are as defined as for Formula I, Y⁴ is hydrogen or deuterium and P² is a protecting group. In one embodiment, Y⁴ is hydrogen. In one embodiment, Y⁴ is deuterium. In one embodiment, Z⁵ is deuterium. In one aspect of this embodiment, Z³ and Z⁴ are each hydrogen. In another aspect of this embodiment, Z³ and Z⁴ are each deuterium. In one aspect of this embodiment, Z¹ and Z² are each hydrogen. In one aspect of this embodiment, Z¹ and Z² are each deuterium. In one more particular aspect, each Z is deuterium.

In one aspect of the embodiment wherein Z⁵ is deuterium, Z³ and Z⁴ are each deuterium; and Z¹ and Z² are each hydrogen.

In one aspect of the embodiment wherein Z⁵ is deuterium, Z³ and Z⁴ are each deuterium; and Z¹ and Z² are each deuterium.

In one aspect of any of the foregoing embodiments or aspects, P² is a group of the formula —C(O)-Q-R², wherein Q is O or NH, and R² is (a) C₁-C₆ alkyl optionally substituted with C₆-C₁₀ aryl; (b) C₃-C₈ cycloalkyl; or (c) C₆-C₁₀ aryl. In one more particular aspect, P² is benzyloxycarbonyl.

In one embodiment, the invention is directed to a compound of formula IV

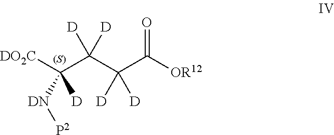

wherein R¹² is C₁-C₆ alkyl and P² is a group of the formula —C(O)-Q-R², wherein Q is O or NH, and R² is (a) C₁-C₆ alkyl optionally substituted with C₆-C₁₀ aryl; (b) C₃-C₈ cycloalkyl; or (c) C₆-C₁₀ aryl. In one aspect, P² is benzyloxycarbonyl. In one aspect R¹² is CH₃.

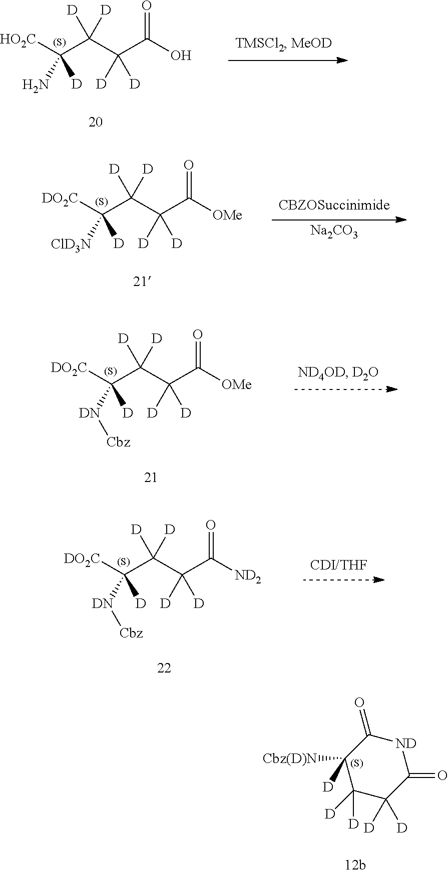

In one embodiment, the invention is directed to a process comprising treating a compound of formula 20

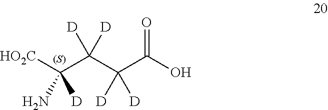

with a silyl halide and a compound of formula R¹²OD to provide a compound of formula 21″:

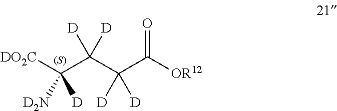

or salt thereof, wherein R¹² is C₁-C₆ alkyl. The silyl halide may be a compound of formula (R¹¹)₃Si-Hal, wherein R¹¹ is C₁-C₆ alkyl and Hal is fluoro, chloro, bromo or iodo. In one aspect of this embodiment, the silyl halide is trimethylsilyl chloride.

In one embodiment, the invention is directed to a process comprising treating a compound of formula IV

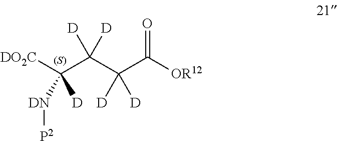

with ND₃ to provide a compound of formula 22:

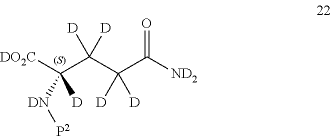

In another set of embodiments, any atom not designated as deuterium in any of the foregoing embodiments or aspects or examples is present at its natural isotopic abundance.

The synthesis of compounds disclosed herein can be readily achieved by synthetic chemists of ordinary skill by reference to the Exemplary Synthesis and Examples disclosed herein. Relevant procedures and intermediates are disclosed, for instance, in U.S. Pat. No. 5,635,517 and US Patent Application 2004147558, in addition to Muller, G W et al., Bioorg Med Chem Lett, 1999, June 7, 9(11): 1625-1630.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula I is depicted in Schemes 1 and 2.

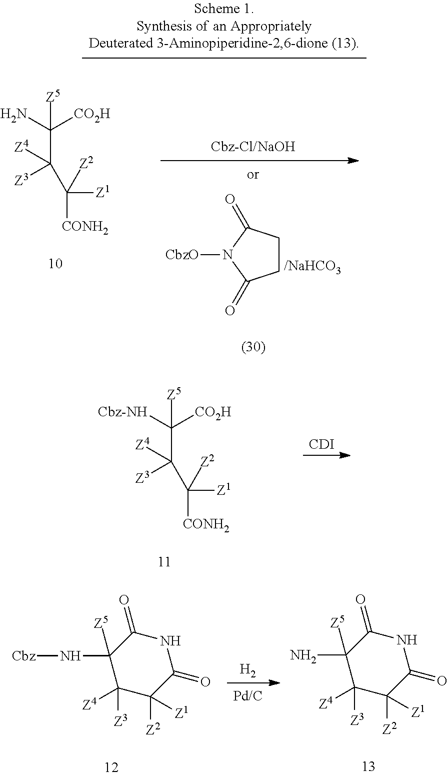

As shown in Scheme 1, reaction of an appropriately deuterated D,L-glutamine 10 (see below) with Cbz-chloride or with compound 30 yields the carbamate 11, which is then cyclized with 1,1′-carbonyldiimidazole (CDI) to yield 12. The carbamate protecting group is then removed from 12 by hydrogenolysis to provide the appropriately deuterated 3-aminopiperidine-2,6-dione 13. This amine is then used as shown in Scheme 2 to produce a compound of Formula I.

Appropriately deuterated D,L-glutamine 10 for use in Scheme 1 above may be prepared, for example, from the corresponding commercially available deuterated glutamic acids (D,L)-2,3,3,4,4-d₅-glutamic acid, (D,L)-2,4,4-d₃-glutamic acid, or (D,L)-3,3-d₂-glutamic acid by methods analogous to those employed by Ogrel, A. et al., Russian Journal of Organic Chemistry, 2001, 37(4): 475-479

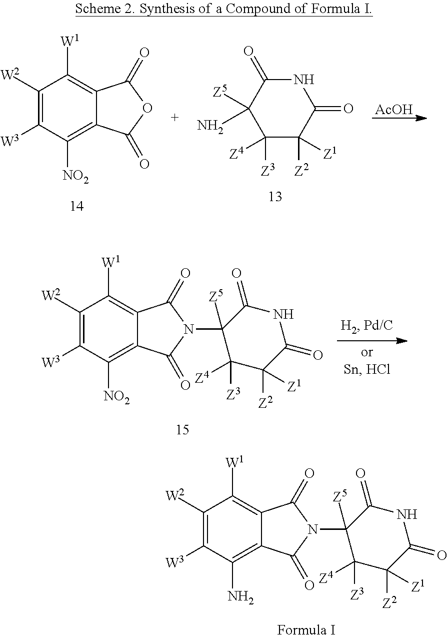

Scheme 2 depicts the preparation of appropriately deuterated compounds of Formula I. Condensation of appropriately deuterated 3-nitrophthalic anhydride 14 (commercially available for W¹, W² and W³=H (14a), see Scheme 4 below for W¹, W² and W³=D (14b)) with appropriately deuterated aminoglutarimide 13 in acetic acid affords the intermediate, 15. Reduction of 15 via catalytic hydrogenation or in the presence of tin and HCl yields the desired compounds of Formula I.

If desired, the R and S enantiomers of a compound of Formula I can then be separated by chiral HPLC in a manner similar to that known for related compounds in the IMiD class of drugs. Examples of this type of chiral HPLC enantiomer separation are found in Sembongi, K. et al., Biological & Pharmaceutical Bulletin, 2008, 31(3): 497-500; Murphy-Poulton, S. F. et al., Journal of Chromatography, B: Analytical Technologies in the Biomedical and Life Sciences, 2006, 831(1-2): 48-56; Eriksson, T. et al., Journal of Pharmacy and Pharmacology, 2000, 52(7): 807-817; Eriksson, T. et al., Chirality, 1998, 10(3): 223-228; Reepmeyer, J. C. et al., Chirality, 1996, 8(1): 11-17; Aboul-Enein, H. Y. et al., Journal of Liquid Chromatography, 1991, 14(4): 667-73; and Teo, S. K. et al., Chirality, 2003, 15(4): 348-351.

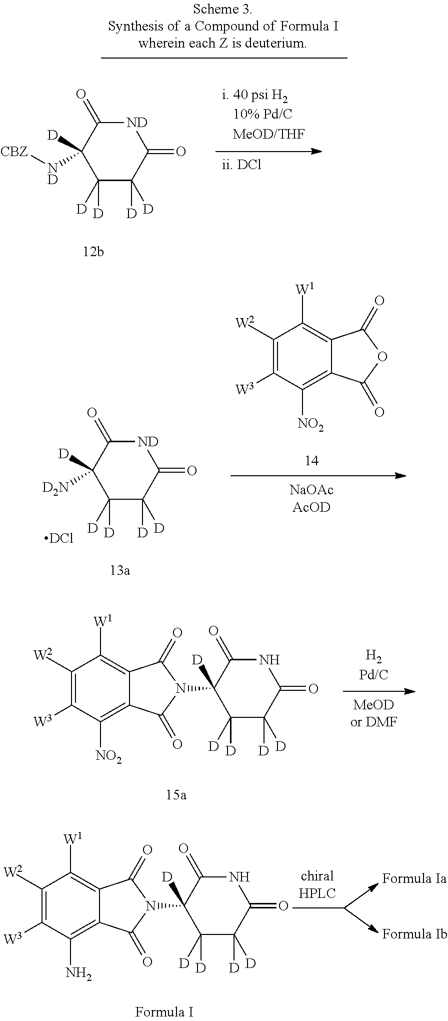

Scheme 3 depicts the preparation of compounds of Formula I wherein each Z is deuterium. Deuterated aminoglutarimide 13a is prepared via catalytic hydrogenation with palladium over carbon of the protected deuterated 3-Aminopiperidine-2,6-dione 12b, an exemplary preparation of which is disclosed in Scheme 5 below. Condensation of appropriately deuterated 3-nitrophthalic anhydride 14 with 13a in AcOD/sodium acetate affords the intermediate, 15a. Reduction of 15a via catalytic hydrogenation with palladium over carbon yields the desired compounds of Formula I. Optionally, the compounds of formula I may be separated into compounds of formula Ia and of formula Ib by chiral HPLC. Intermediates 12b and 18 may also be prepared as discussed in published application WO 2010/056344, incorporated herein in its entirety.

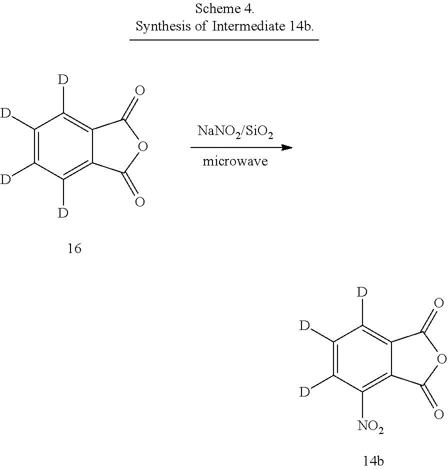 [SMILES: [2H]c1c([2H])c([2H])c2c(c1[2H])C(=O)OC2=O ; [2H]c1c([2H])c2c(c([N+](=O)[O-])c1[2H])C(=O)OC2=O]

Scheme 4 depicts the preparation of 3-nitrophthalic anhydride-d₃ (14b) for use in Scheme 2 above. Treatment of commercially available isobenzofurandione-4,5,6,7-d₄ (16) under microwave irradiation with sodium nitrite adsorbed onto silica gel in a manner analogous to that described by Badgujar, D M et al., J. of Scientific and Industrial Research, 2007, 66(3): 250-251 affords 14b. Alternatively, treatment of 16 with sulfuric acid and nitric acid in a manner analogous to that described by Chen, Zhi-min; et al., Hecheng Huaxue (2004), 12(2), 167-169, 173; or by Murthy, Y. L. N.; et al., Oriental Journal of Chemistry (2009), 25(2), 299-306, affords 14b.

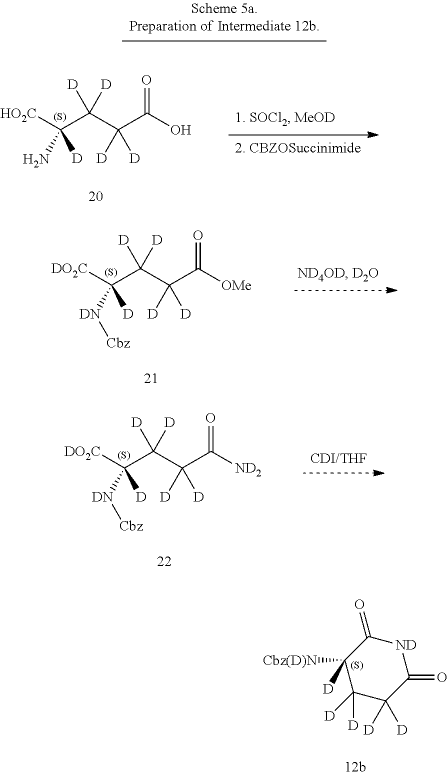

Scheme 5a depicts a preparation of the protected deuterated 3-Aminopiperidine-2,6-dione 12b. Deuterated glutamic acid 20, an exemplary preparation of which is shown in Scheme 6 below, is treated with SOCl₂ and CH₃OD followed by N-(benzyloxycarbonyloxy)succinimide to provide 21. Reaction of 21 with ammonia-d5 in D₂O gave amide 22 which upon treatment with carbonyldiimidazole (CDI) cyclized to 12b.

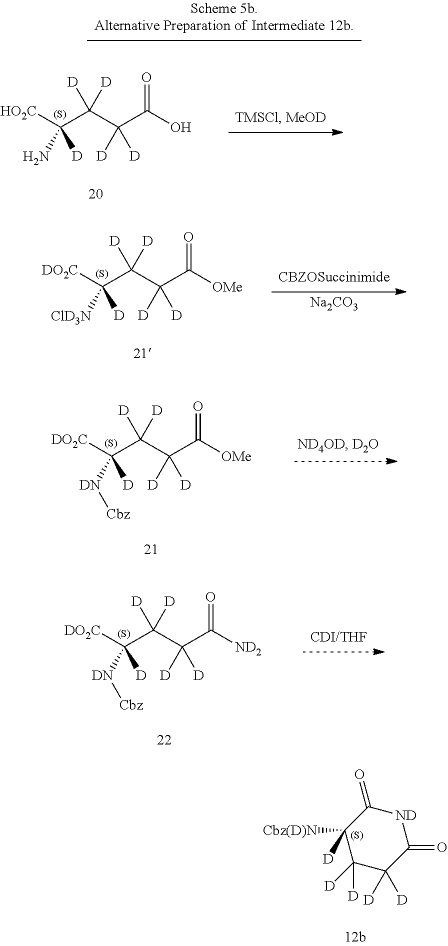

Scheme 5b depicts an alternative preparation of 12b. Deuterated glutamic acid 20 is treated with TMSCl (2.2 equivalents) in CH₃OD to give 21′ which is treated with N-(benzyloxycarbonyloxy)succinimide and sodium carbonate (2 equivalents) to provide 21. Reaction of 21 with deuterated ammonia in D₂O gave amide 22 which upon treatment with carbonyldiimidazole (CDI) cyclized to 12b.

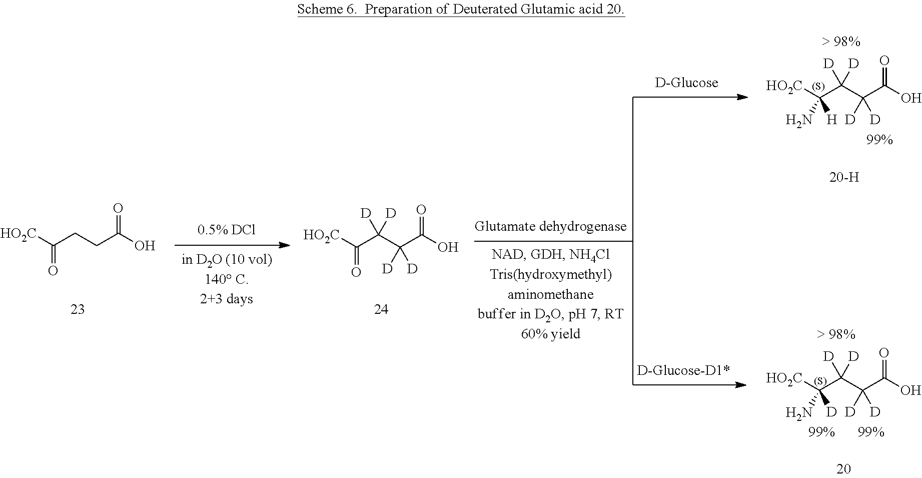

Scheme 6 depicts a preparation of deuterated glutamic acid 20. Succinic acid 23 was treated with DCl in D₂O to provide 24, which was treated with D-glucose-D₁ NAD (Nicotinamide adenine dinucleotide). D-glucose-D₁ is the following compound (shown below in its open chain and pyranose forms):

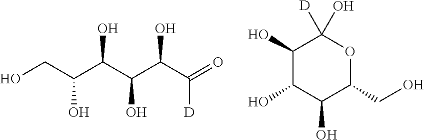 [SMILES: [2H]C(=O)[C@H](O)[C@@H](O)[C@H](O)[C@H](O)CO ; [2H]C1(O)O[C@H](CO)[C@@H](O)[C@H](O)[C@H]1O]

More generally, 24 may be treated with a deuteride source (to provide 20) or a hydride source (to provide 20-H), where the deuteride or hydride source is a compound or mixture capable of providing a deuteride or hydride anion, respectively, or the synthetic equivalent thereof. Such mixture may comprise a co-factor, an example of which is NAD as illustrated in Scheme 6. Another example of a co-factor is NADP. The mixture may also comprise a co-factor regeneration system, which may comprise, as an example, a dehydrogenase and a substrate for the dehydrogenase. In the example shown in Scheme 6, the mixture comprises GDH as the dehydrogenase; D-Glucose-D₁ (to produce 20) or D-glucose (to produce 20-H) as the substrate; and NAD as the co-factor. In one embodiment, the D-glucose-D₁ is generated in situ from inexpensive D-glucono-δ-lactone and NaBD₄. This embodiment is advantageous in that an otherwise expensive deuterated glucose substrate is generated from relatively inexpensive reagents. Other embodiments of the deuteride or hydride source are disclosed in paragraphs [43[-[53] of application PCT/US2011/050138, and in the corresponding paragraphs of U.S. provisional application 61/379,182, incorporated by reference herein in their entirety. The isotopic enrichment factor in 20 and 20-H is over 98% at each of the positions designated with deuterium in the two structures.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of the formulae herein and their synthetic precursors, including those within routes not explicitly shown in Schemes herein, are within the means of chemists of ordinary skill in the art. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I (e.g., including any of the formulae herein), or a pharmaceutically acceptable salt thereof; and an acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Such administration is known to be effective with erectile dysfunction drugs: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein. In one preferred embodiment, a compound of Formula I is formulated into a hydrogel for delivery to the eye as described in United States Patent Publication US2005074497.

According to another embodiment, the invention provides a method of coating an implantable medical device comprising the step of contacting said device with the coating composition described above. It will be obvious to those skilled in the art that the coating of the device will occur prior to implantation into a mammal

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with an immunomodulator, an anti-angiogenic or an anti-neoplastic agent. Such agents are described in detail in U.S. Pat. No. 5,635,517, as well as in PCT patent publications WO2005097125, WO2005055929, WO2004041190, WO2006060507, WO2006058008, WO2006053160, WO2005044178, WO2004100953, WO2006089150, WO2006036892, WO2006018182, WO2005082415, WO2005048942, WO2005042558, WO2005035714 and WO2005027842; and in United States Patent publications US2005100529, US2006030594, US2005143344 and US2006079461, each of the foregoing of which describes second therapeutic agents that may be combined with pomalidomide.

In one embodiment, the second therapeutic agent is an agent useful in the treatment or prevention of a disease or condition selected from myelodysplastic syndromes, multiple myeloma, Non-Hodgkins lymphoma; papillary and follicular thyroid carcinoma; chronic lymphocytic leukemia, amyloidosis, complex regional pain syndrome Type I, malignant melanoma, radiculopathy, glioblastoma, gliosarcoma, malignant gliomas, myelogenous leukemia, refractory plasma cell neoplasm, chronic myelomonocytic leukemia, follicular lymphoma, ciliary body and chronic melanoma, iris melanoma, recurrent interocular melanoma, extraocular extension melanoma, solid tumors, T-cell lymphoma, erythroid lymphoma, monoblastic and monocytic leukemia; myeloid leukemia, brain tumor, meningioma, spinal cord tumors, thyroid cancers, mantle cell lymphoma, non-small cell lung cancer, ovarian cancer, prostate cancer, renal cell cancer, myelofibrosis, Burkitt's lymphoma, Hodgkin's lymphoma, large cell lymphoma, pancreatic cancer, idiopathic pulmonary fibrosis, graft vs. host disease, soft tissue sarcoma, small cell lung cancer or Waldenstrom's macroglobulinemia.

In another embodiment, the second therapeutic agent is an agent useful in the treatment or prevention of a disease or condition selected from dysfunctional sleep, hemoglobinopathy, anemia, macular degeneration, atherosclerosis, restenosis, pain, immunodeficiencies, CNS injury and related symptoms, CNS disorders, parasitic disease, or asbestos-related disease.

Even more preferably the second therapeutic agent co-formulated with a compound of this invention is an agent useful in the treatment of myelodysplastic syndromes or multiple myeloma.

In another preferred embodiment, the second therapeutic agent is selected from aldesleukin; a p38 MAP kinase inhibitor such as disclosed in US2006079461; a 24-hydroxylase inhibitor such as disclosed in WO2006036892; an aminopteridinone such as disclosed in WO2006018182; an IGF-R inhibitor such as disclosed in WO2005082415; a COX-2 inhibitor such as disclosed in WO2005048942; a nucleobase oligomer such as disclosed in WO2005042558; a chlorpromazine compound such as disclosed in WO2005027842.

In yet another preferred embodiment, the second therapeutic agent is selected from pemetrexed, topotecan, doxorubicin, bortezomib, gemcitabine, dacarbazine, dexamethasone, clarithromycin, doxil, vincristine, decadron, azacitidine, rituximab, prednisone, docetaxel, melphalan, cyclophosphamide, cisplatin, etoposide and combinations thereof.

In yet another preferred embodiment, the second therapeutic agent is selected from bortezomib, gemcitabine, dexamethasone, clarithromycin, rituximab, prednisone, cyclophosphamide, cisplatin, etoposide and combinations thereof.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., 1966, Cancer Chemother Rep, 50: 219. Body surface area may be approximately determined from height and weight of the patient. See, e.g., Scientific Tables, Geigy Pharmaceuticals, Ardsley, N.Y., 1970, 537. An effective amount of a compound of this invention can range from about 0.005 mg/kg to about 200 mg/kg, more preferably 0.01 mg/kg to about 100 mg/kg, more preferably 0.05 mg/kg to about 60 mg/kg.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for pomalidomide.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the second therapeutic agents referenced above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

According to another embodiment, the invention provides a method of treating a disease that is beneficially treated by pomalidomide in a patient in need thereof, comprising the step of administering to the patient an effective amount of a compound or a composition of this invention. Such diseases are well known in the art and are disclosed in U.S. Pat. No. 5,635,517, as well as in PCT patent publications WO2005097125, WO2005055929, WO2004041190, WO2006060507, WO2006058008, WO2006053160, WO2005044178, WO2004100953, WO2006089150, WO2006036892, WO2006018182, WO2005082415, WO2005048942, WO2005042558, WO2005035714 and WO2005027842; and in United States Patent publications US2005100529, US2006030594, US2005143344 and US2006079461.

In one preferred embodiment, the disease or condition is selected from myelodysplastic syndromes, multiple myeloma, Non-Hodgkins lymphoma; papillary and follicular thyroid carcinoma; chronic lymphocytic leukemia, amyloidosis, complex regional pain syndrome Type I, malignant melanoma, radiculopathy, glioblastoma, gliosarcoma, malignant gliomas, myelogenous leukemia, refractory plasma cell neoplasm, chronic myelomonocytic leukemia, follicular lymphoma, ciliary body and chronic melanoma, iris melanoma, recurrent interocular melanoma, extraocular extension melanoma, solid tumors, T-cell lymphoma, erythroid lymphoma, monoblastic and monocytic leukemia; myeloid leukemia, brain tumor, meningioma, spinal cord tumors, thyroid cancers, mantle cell lymphoma, non-small cell lung cancer, ovarian cancer, prostate cancer, renal cell cancer, myelofibrosis, Burkitt's lymphoma, Hodgkin's lymphoma, large cell lymphoma, pancreatic cancer, idiopathic pulmonary fibrosis, graft vs. host disease, soft tissue sarcoma, solid tumors, small cell lung cancer or Waldenstrom's macroglobulinemia.

In another embodiment, the disease is selected from myelodysplastic syndromes or multiple myeloma.

Identifying a patient in need of such treatment can be in the judgment of a patient or a health care professional and can be subjective (e.g. opinion) or objective (e.g. measurable by a test or diagnostic method).

In another embodiment, the above method of treatment comprises the further step of co-administering to the patient one or more second therapeutic agents. The choice of second therapeutic agent may be made from any second therapeutic agent known to be useful for co-administration with pomalidomide. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In one embodiment, the second therapeutic agent and the corresponding disease for which the second therapeutic agent is co-administered with a compound of this invention is set forth in Table 1 below.

TABLE 1
Second Therapeutic Agents for Various Diseases or Conditions

| Second Therapeutic Agent | Disease or Condition |
|---|---|
| Irinotecan | Multiple myeloma |
| Aldesleukin | Tumor prevention or treatment |
| P38 MAP kinase inhibitor | Multiple myeloma |
| 24-hydroxylase inhibitor | Cancer |
| Aminopteridinone | Cancer |
| IGF-1R inhibitor | Tumor treatment |
| COX-2 inhibitor | Neoplasia |
| Nucleobase oligomer | Neoplasia |
| Chlorpromazine | Neoplasia |
| Pemetrexed | Non-small cell lung cancer |
| Topotecan | ovarian and primary peritoneal |
| | carcinoma |
| doxorubicin | ovarian and primary peritoneal |
| | carcinoma |
| doxorubicin and dexamethasone | multiple myeloma |
| Bortezomib | multiple myeloma |
| Bortezomib and dexamethasone | multiple myeloma |
| dexamethasone | multiple myeloma |
| Gemcitabine | pancreatic cancer |
| DTIC (Dacarbazine) | Malignant myeloma |
| DVd (Doxil, Vincristine and | multiple myeloma |
| Decadron) | |
| azacitidine | myelodysplastic syndrome |
| radiation therapy | glioblastoma, gliosarcoma, |
| | malignant glioma |
| Rituximab | chronic lymphocytic leukemia, |
| | follicular lymphoma, mantle cell |
| | lymphoma, Waldenstrom's |
| | Macroglobulinemia |
| prednisone | Myelofibrosis |
| docetaxel | solid tumor |
| melphalan | multiple myeloma |
| Cyclophosphamide and prednisone | multiple myeloma |
| Clarithromycin and dexamethasone | multiple myeloma |
| Rituximab and dexamethasone | Waldenstrom's Macroglobulinemia |
| Cisplatin | Small cell lung cancer |
| etoposide | Small cell lung cancer |

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a patient does not preclude the separate administration of that same therapeutic agent, any other second therapeutic agent or any compound of this invention to the patient at another time during a course of treatment.

Effective amounts of these second therapeutic agents are well known to those skilled in the art and guidance for dosing may be found in patents and published patent applications referenced herein, as well as in Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention where a second therapeutic agent is administered to a patient, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above.

Another aspect of the invention is a compound of Formula I for use in the treatment or prevention in a patient of a disease, disorder or symptom thereof delineated herein.

### EXAMPLES

### Example 1

### Synthesis of (S)-3-(Amino-d₂)(piperidine-1,3,4,4,5,5-d₆)-2,6-dione deuterium chloride salt (13a)

Intermediate 13a was prepared as outlined in Scheme 7 below, following the experimental procedure disclosed in patent publication WO 2010/056344 paragraphs [98]-[100]. Alternatively, intermediate 13a was prepared as outlined in Scheme 8 below, or according to Scheme 3 above.

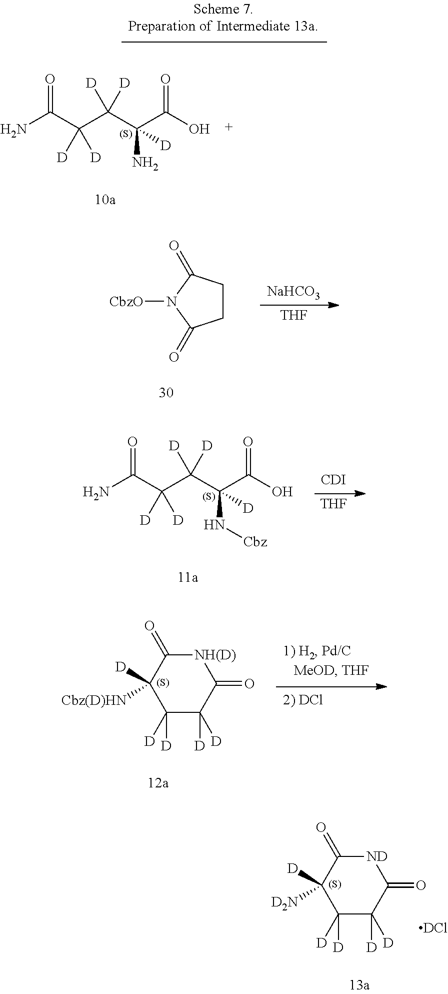

Intermediate 13a is then converted to a compound of Formula I in accordance with Scheme 2 or Scheme 3. For example, compound 101a (containing an amount of compound 101b smaller than the amount of compound 101a) of formula I was prepared as disclosed in Scheme 8:

### Example 2

### Synthesis of (S)-4-Amino-2-(3,4,4,5,5-d₅-2,6-dioxopiperidin-3-yl)isoindoline-1,3-dione (101a)

Compound 101a was prepared as outlined in Scheme 8 below:

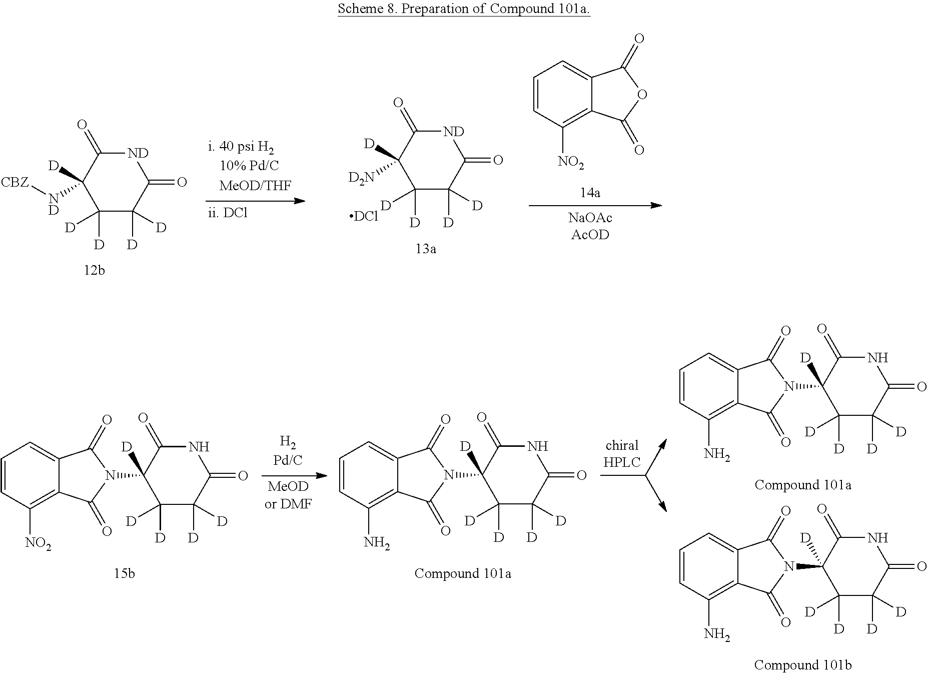

### Step 1. (S)-Benzyl deutero(1,3,4,4,5,5-d₆-2,6-dioxopiperidin-3-yl)carbamate (13a)

A solution of 12b (300 mg, 1.1 mmol, prepared as outlined in Example 1 above) in tetrahydrofuran (10 mL) and methanol-D (10 mL) was added to 10% Pd/C (50% wet with D₂O, CIL, 99.9 atom % D) and hydrogenated using a Parr shaker at 40 psi H₂ for 5 hours. The mixture was filtered through a pad of Celite (washing with THF). To the filtrate was added 0.3 mL of a 35% solution of deuterium chloride in D₂O (Aldrich, 99 atom % D) resulting in a white suspension. The solvent was evaporated yielding 13a as an off white solid (210 mg, quantitative).

### Step 2. (S)-4-Nitro-2-(3,4,4,5,5-d₅-2,6-dioxopiperidin-3-yl)isoindoline-1,3-dione (15b)

A 10 mL microwave vial was charged with 13a (150 mg, 0.87 mmol, 1 equiv), 3-nitrophthalic anhydride (165 mg, 0.87 mmol, 1 equiv) and anhydrous sodium acetate (91 mg, 1.3 mmol, 1.5 equiv). Acetic acid-D (2 mL, Aldrich, 99 atom % D) was added and the reaction was heated by microwave irradiation for 3 hours at 115° C. The resulting dark purple/black suspension was filtered, washed with acetic acid-D (6 mL), D₂O (25 mL, Cambridge Isotopes, 99 atom % D), and MTBE (25 mL). The resulting purple/grey solid was dried in a vacuum oven at 40° C. for 16 hours yielding 15b as a purple/grey solid (76 mg, 30% yield). m/z=309 (M+H)⁺, 331 (M+Na)⁺.

### Step 3. (S)-4-Amino-2-(3,4,4,5,5-d₅-2,6-dioxopiperidin-3-yl)isoindoline-1,3-dione (101a)

A solution of 15b (70 mg, 0.23 mmol) in DMF (10 mL) was hydrogenated over 10% Pd/C (9 mg, 50% wet with D₂O) for 3 hours at 35 psi H₂, at which time LC/MS indicated complete consumption of 15b and the formation of 101a (90%, m/z=279 for d5 M+H) along with the hydroxylamine intermediate (˜10%). The hydrogenation was continued an additional hour resulting in complete conversion to 101a. The reaction mixture was then filtered through a pad of celite, washing with THF. The filtrate was then evaporated giving a yellow-brown residue. The residue was suspended in ethyl acetate/THF (20 mL/10 mL) and heated to reflux. The suspension was filtered hot and the filtrate was concentrated to ˜5 mL. Heptane (10 mL) was added and the solids were collected via vacuum filtration yielding 101a as a yellow/green solid (36 mg, 56% yield). m/z=279 (M+H)⁺, 301 (M+Na)⁺.

A smaller amount of Compound 101b may be formed in addition to Compound 101a under the reaction conditions. Compounds 101a and 101b may be separated and isolated using chiral HPLC.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

We claim:

1-18. (canceled)

19. A compound of Formula I: 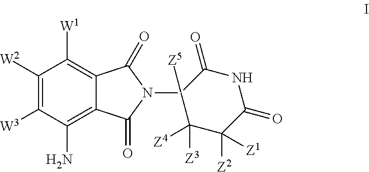
or a pharmaceutically acceptable salt thereof, wherein:
Z⁵ is deuterium;
each W is independently selected from hydrogen or deuterium;
each Z is independently selected from hydrogen or deuterium; and
at least one W or one Z is deuterium.

20. The compound of claim 19, wherein the compound is 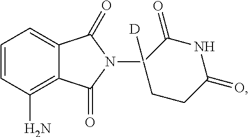 [SMILES: [2H]C1(N2C(=O)c3cccc(N)c3C2=O)CCC(=O)NC1=O]
or a pharmaceutically acceptable salt thereof, wherein any atom not designated as deuterium is present at its natural isotopic abundance.

21. The compound of claim 19, wherein the compound is of formula I and is selected from:
Z¹ and Z² are each deuterium;
Z¹, Z², Z³ and Z⁴ are simultaneously deuterium;
W¹, W² and W³ are simultaneously deuterium;
Z¹, Z², Z³, Z⁴, W¹, W² and W³ are deuterium; and
Z¹, Z², W¹, W² and W³ are deuterium;
or a pharmaceutically acceptable salt thereof, wherein the non-specified groups are selected from hydrogen and deuterium.

22. The compound of claim 19, wherein the compound is of formula I and is selected from:
Z¹ and Z² are each deuterium; and Z³, Z⁴, W¹, W² and W³ are hydrogen;
Z¹, Z², Z³ and Z⁴ are each deuterium and W¹, W² and W³ are hydrogen;
W¹, W² and W³ are deuterium and Z¹, Z², Z³ and Z⁴ are hydrogen;
Z¹, Z², Z³, Z⁴, W¹, W² and W³ are deuterium; and
Z¹, Z², W¹, W² and W³ are deuterium and Z³ and Z⁴ are hydrogen;
or a pharmaceutically acceptable salt thereof.

23. A compound of Formula Ib: 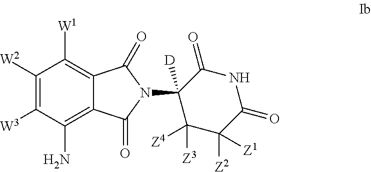
or a pharmaceutically acceptable salt thereof, wherein:
each W is independently selected from hydrogen or deuterium; and
each Z is independently selected from hydrogen or deuterium.

24. The compound of claim 23, wherein the compound contains less than 25% of the other enantiomer.

25. The compound of claim 23, wherein the compound is 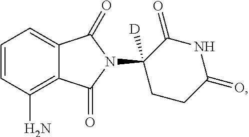 [SMILES: [2H][C@@]1(N2C(=O)c3cccc(N)c3C2=O)CCC(=O)NC1=O]
or a pharmaceutically acceptable salt thereof, wherein any atom not designated as deuterium is present at its natural isotopic abundance.

26. The compound of claim 23, wherein the compound is of formula Ib and is selected from:
Z¹ and Z² are each deuterium;
Z¹, Z², Z³ and Z⁴ are each deuterium;
W¹, W² and W³ are simultaneously deuterium;
Z¹, Z², Z³, Z⁴, W¹, W² and W³ are deuterium; and
Z¹, Z², W¹, W² and W³ are deuterium;
or stereoisomer or pharmaceutically acceptable salt thereof, wherein the non-specified groups are selected from hydrogen and deuterium.

27. The compound of claim 23, wherein the compound is of formula I and is selected from:
Z¹ and Z² are each deuterium; and Z³, Z⁴, W¹, W² and W³ are hydrogen;
Z¹, Z², Z³ and Z⁴ are each deuterium and W¹, W² and W³ are hydrogen;
W¹, W² and W³ are deuterium and Z¹, Z², Z³ and Z⁴ are hydrogen;
Z¹, Z², Z³, Z⁴, W¹, W² and W³ are deuterium; and
Z¹, Z², W¹, W² and W³ are deuterium and Z³ and Z⁴ are hydrogen;
or a pharmaceutically acceptable salt thereof.

28. A compound of Formula Ia: 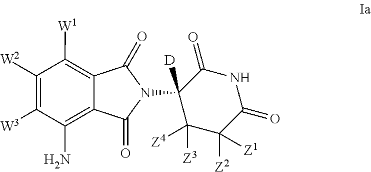
or a pharmaceutically acceptable salt thereof, wherein:
each W is independently selected from hydrogen or deuterium; and
each Z is independently selected from hydrogen or deuterium.

29. The compound of claim 28, wherein the compound contains less than 25% of the other enantiomer.

30. The compound of claim 28, wherein the compound is 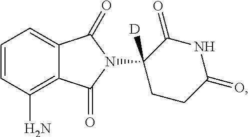 [SMILES: [2H][C@]1(N2C(=O)c3cccc(N)c3C2=O)CCC(=O)NC1=O]
or a pharmaceutically acceptable salt thereof wherein any atom not designated as deuterium is present at its natural isotopic abundance.

31. The compound of claim 28, wherein the compound is of formula Ib and is selected from:
Z¹ and Z² are each deuterium;
Z¹, Z², Z³ and Z⁴ are each deuterium;
W¹, W² and W³ are simultaneously deuterium;
Z¹, Z², Z³, Z⁴, W¹, W² and W³ are deuterium; and
Z¹, Z², W¹, W² and W³ are deuterium;
or stereoisomer or pharmaceutically acceptable salt thereof, wherein the non-specified groups are selected from hydrogen and deuterium.

32. The compound of claim 28, wherein the compound is of formula I and is selected from:
Z¹ and Z² are each deuterium; and Z³, Z⁴, W¹, W² and W³ are hydrogen;
Z¹, Z², Z³ and Z⁴ are each deuterium and W¹, W² and W³ are hydrogen;
W¹, W² and W³ are deuterium and Z¹, Z², Z³ and Z⁴ are hydrogen;
Z¹, Z², Z³, Z⁴, W¹, W² and W³ are deuterium; and
Z¹, Z², W¹, W² and W³ are deuterium and Z³ and Z⁴ are hydrogen;
or a pharmaceutically acceptable salt thereof.

33. A pharmaceutical composition comprising a compound of claim 19 or a pharmaceutically acceptable salt thereof and a pharmaceutically acceptable carrier.

34. A pharmaceutical composition comprising a compound of claim 23 or a pharmaceutically acceptable salt thereof and a pharmaceutically acceptable carrier.

35. A pharmaceutical composition comprising a compound of claim 28 or a pharmaceutically acceptable salt thereof and a pharmaceutically acceptable carrier.

36. A method of treating multiple myeloma in a patient in need thereof, the method comprising administering to the patient a compound of claim 19 or a pharmaceutically acceptable salt thereof.

37. A method of treating multiple myeloma in a patient in need thereof, the method comprising administering to the patient a compound of claim 23 or a pharmaceutically acceptable salt thereof.

38. A method of treating multiple myeloma in a patient in need thereof, the method comprising administering to the patient a compound of claim 28 or a pharmaceutically acceptable salt thereof.
